(19) United States
(12) Patent Application Publication
(10) Pub. No.: US 2009/0076465 A1
(43) Pub. Date: Mar. 19, 2009

(54) COMPOSITE SEAL AND METHOD FOR MANUFACTURING

(75) Inventors: Danny Berry, Hamden, CT (US); Michael Bettuchi, Middletown, CT (US); Richard D. Gresham, Guilford, CT (US); Steven L. Izzo, Naugatuck, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/174,736

(22) Filed: Jul. 17, 2008

Related U.S. Application Data
(60) Provisional application No. 60/994,048, filed on Sep. 17, 2007.

(51) Int. Cl. A61L 29/14; A61M 25/00; B23P 19/04

(52) U.S. Cl. 604/265; 604/523; 29/460

(57) ABSTRACT

The present disclosure provides a composite surgical seal for use in a surgical access device which defines an access channel through it and includes a seal member configured and dimensioned to form a seal with a housing interior wall of a surgical access device. The seal member includes a layer defining an orifice therethrough and a fabric layer substantially encapsulating the resilient layer such that a surface of the resilient layer which defines the orifice is covered by the fabric layer. The access channel is configured and dimensioned such that insertion of a surgical instrument into the access channel causes the seal member to form a substantial sealing relation with the surgical instrument inserted therethrough. A method of forming a composite surgical seal in accordance with the present disclosure is also provided herewith.

### CROSS REFERENCE TO RELATED APPLICATION

The present application claims the benefit of and priority to U.S. Provisional Application Ser. No. 60/994,048 filed on Sep. 17, 2007, the entire contents of which are incorporated herein by reference.

### BACKGROUND

1. Technical Field

The present disclosure relates to surgical devices and, more particularly, relates to a seal assembly for use with a surgical access device during a minimally invasive surgical procedure, for example, in both laparoscopic and endoscopic procedures.

2. Description of Related Art

Minimally invasive surgical procedures avoid open invasive surgery in favor of closed or local surgery with less trauma. These procedures involve use of laparoscopic devices and remote-control manipulation of instruments with indirect observation of the surgical field through an endoscope or similar device, and are carried out through the skin or through a body cavity or anatomical opening. Laparoscopic and endoscopic procedures generally require that any instrumentation inserted into the body be sealed, i.e. provisions must be made to ensure that gases do not enter or exit the body through the incision as, for example, in surgical procedures in which the surgical region is insufflated. These procedures typically employ surgical instruments which are introduced into the body through a cannula. The cannula has a seal assembly associated therewith and provides a substantially fluid tight seal about the instrument to preserve the integrity of the established air or gas within the surgical region.

Minimally invasive procedures have several advantages over traditional open surgery, including less patient trauma, reduced recovery time, reduced potential for infection, etc. However, minimally invasive surgery, such as laparoscopy, has several disadvantages. In particular, the frictional forces exerted on surgical instruments inserted through it, has proved to be difficult in procedures requiring extensive manipulation of the long narrow endoscopic instruments within a remote site because of the restricted mobility. In addition, known seal devices are deficient in resilience and in rigidity for affixing the seal within a cannula or trocar housing.

### SUMMARY

A surgical access device is provided which includes a housing having an interior wall defining a longitudinal axis and having at least one aperture configured and dimensioned to permit passage of a surgical instrument through the aperture. The surgical access device includes a seal member supported in the housing and defining an access channel through the seal member. The seal member includes a resilient layer forming a seal with the housing interior wall and defining an orifice through it. The seal member also includes a fabric layer substantially encapsulating the resilient layer such that a surface of the resilient layer which defines the orifice is covered by the fabric layer. The access channel is configured and dimensioned such that insertion of a surgical instrument into the access channel causes the seal member to form a substantial sealing relation with the surgical instrument when it is inserted therethrough.

A composite surgical seal is provided for use in a surgical access device defining an access channel through the seal, and includes a seal member configured and dimensioned to form a seal with a housing interior wall of a surgical access device. The seal member includes a resilient layer defining an orifice therethrough and a fabric layer substantially encapsulating the resilient layer such that a surface of the resilient layer which defines the orifice is covered by the fabric layer. The access channel is configured and dimensioned such that insertion of a surgical instrument into the access channel causes the seal member to form a substantial sealing relation with the surgical instrument inserted therethrough.

A method of forming a composite seal assembly for use in a surgical access device is also provided whereby the steps include initially providing first and second fabric ring assemblies each including a rigid ring having a fabric layer secured to it. The first and second fabric ring assemblies are then positioned in opposing relation to each other such that a gap is created between them preceding approximation of opposing central portions of each fabric layer. Subsequently, a gel material is introduced between the first and second fabric layers to fill the gap formed between the first and second fabric ring assemblies to form the seal assembly. An orifice is then formed through a central portion of the seal assembly such that the fabric layer of one or both of the fabric ring assemblies covers the surface of the orifice.

The seal member may also include a rigid ring layer attached to an outer circumference of at least one of a proximal end or distal end of the seal member and is adapted for mounting to the housing.

### BRIEF DESCRIPTION OF THE DRAWINGS

Various embodiments of the present disclosure will be described hereinbelow with reference to the figures wherein:

FIGS. 1 and 2 are perspective views of an access assembly and a seal assembly in accordance with the principles of the present disclosure;

FIG. 3 is a perspective view with parts separated of the access and seal assemblies of FIG. 1;

FIG. 4 is a partial side cross-sectional view of the access and seal assemblies;

FIG. 5 is a perspective view illustrating a seal assembly in accordance with the present disclosure;

FIG. 6A is an enlarged side cross-sectional view of an embodiment of the seal assembly of FIG. 1;

FIG. 6B is an enlarged view of the indicated area of detail of the seal assembly of FIG. 6A;

FIG. 7A is a top plan view of the seal assembly;

FIG. 7B is a side cross sectional view of the seal assembly of FIG. 7A taken along section line A-A; and

FIG. 8 is a flow chart illustrating the steps of a method for forming a seal assembly in accordance with the present disclosure.

### DETAILED DESCRIPTION

The seal assembly of the present disclosure, either alone or in combination with a seal system internal to a cannula assembly, provides a substantial seal between a body cavity of a patient and the outside atmosphere before, during and after insertion of an object through the cannula assembly. Moreover, the seal assembly of the present disclosure is capable of accommodating objects of varying diameters, e.g., instruments from about 4.5 mm to about 15 mm, while maintaining a fluid tight interface about the instrumentation adapted for insertion through a trocar and/or cannula assembly to prevent gas and/or fluid leakage so as to preserve the atmospheric integrity of a surgical procedure. The flexibility of the present seal assembly greatly facilitates endoscopic and/or laparoscopic surgery where a variety of instruments having differing diameters are often needed during a single surgical procedure. Specifically, the surgical device includes a seal assembly which facilitates lateral and/or angular manipulation of the surgical instrument while also maintaining a seal about the instrument. The seal assembly is further adapted to substantially close in the absence of a surgical instrument to maintain the integrity of the insufflated peritoneal cavity.

The surgical seal assembly of the present disclosure is additionally adapted to decrease the frictional forces exerted on surgical instruments inserted through it which has proven to be difficult in procedures requiring extensive manipulation of the long narrow endoscopic instruments within a remote site because of the restricted mobility.

Moreover, the manufacturing of a durable seal assembly for use with a surgical access device has proven to be expensive and lacking effectiveness with maintaining good seal properties. The present disclosure provides for a more efficient and cost effective way of manufacturing a seal assembly to provide good sealing and durable properties. Specifically, the manufacturing of the seal assembly of the present disclosure provides for effective sealing properties during on and off axis motion while reducing the frictional forces of the surgical instruments lodged therethrough.

Examples of surgical instrumentation include clip appliers, graspers, dissectors, retractors, staplers, laser probes, photographic devices, endoscopes and laparoscopes, tubes, and the like. Such instruments will be collectively referred to herein as “instruments” or “instrumentation”.

The seal assembly may also be adapted dimensionally to receive and form a seal about a physician's arm or hand during a hand-assisted laparoscopic procedure. In this application, the seal assembly is a component of an access member which is introduced within the body to provide access to underlying tissue in, e.g., the abdominal cavity.

Moreover, the seal assembly may be readily incorporated into an access device, such as a conventional trocar device or cannula housing to provide the device with zero-closure and/or sealing around an instrument or other object.

The subject matter of this disclosure generally relates to the subject matter of commonly assigned U.S. provisional application entitled SURGICAL PORTAL WITH GEL AND FABRIC SEAL ASSEMBLY filed under Express Mail Certificate No. EM 075410446 US on Sep. 17, 2007, Attorney Docket No. H-US-00806 (203-5424), the entire contents of which are incorporated herein by reference.

In the following discussion, the term “proximal” or “trailing” will refer to the portion of the surgical device nearest to the clinician during operation while the term “distal” or “leading” will refer to that portion of the portal apparatus most remote to the clinician.

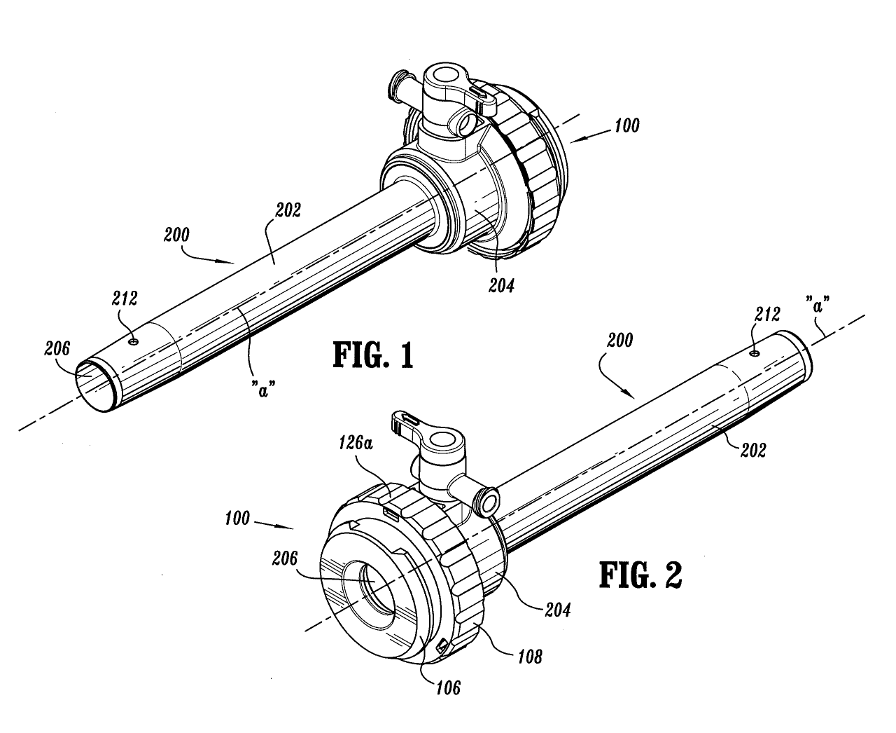

Referring now to the drawings, in which like reference numerals identify identical or substantially similar parts throughout the several views, FIGS. 1-2 illustrate one embodiment of a seal assembly, i.e. seal assembly 100 of the present disclosure mounted to an access device 200 such as cannula or trocar assembly. Cannula assembly 200 may be any conventional cannula suitable for the intended purpose of accessing a body cavity and typically defines a passageway permitting introduction of instruments therethrough. Cannula assembly 200 is particularly adapted for use in laparoscopic surgery where the peritoneal cavity is insufflated with a suitable gas, e.g., CO₂, to raise the cavity wall from the internal organs therein. Cannula assembly 200 is typically used with an obturator assembly (not shown) which may be blunt, a non-bladed, or a sharp pointed instrument positionable within the passageway of the cannula assembly 200. The obturator assembly is utilized to penetrate the abdominal wall or introduce the cannula assembly 200 through the abdominal wall, and then subsequently is removed from the access device to permit introduction of the surgical instrumentation utilized to perform the procedure through the passageway.

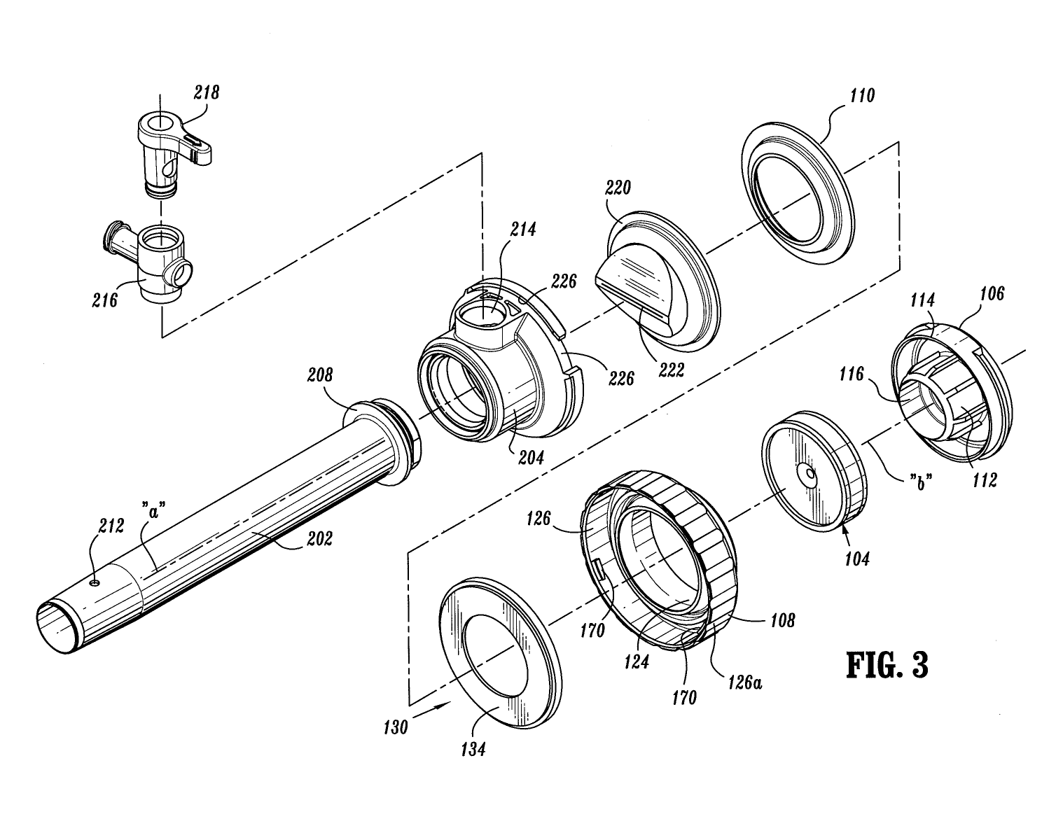

With reference to FIGS. 1-4, cannula assembly 200 includes cannula sleeve 202 and cannula housing 204 mounted to an end of the sleeve 202. Any means for mounting cannula sleeve 202 to cannula housing 204 are envisioned including threaded arrangements, bayonet coupling, snap-fit arrangements, adhesives, etc. Cannula sleeve 202 and cannula housing 204 may be integrally formed. Cannula sleeve 202 defines a longitudinal axis “a” extending along the length of sleeve 202. Sleeve 202 further defines an internal longitudinal passage 206 dimensioned to permit passage of surgical instrumentation. Sleeve 202 defines collar 208 which is mounted to cannula housing 202 and an inner tapered wall 210 adjacent the collar 208. The sloped configuration of tapered wall 210 may assist in guiding the inserted instrument into longitudinal passage 206. Adjacent the distal end of cannula sleeve 202 is aperture 212 which extends through the wall of the sleeve 202. Aperture 212 permits passage of insufflation gases through cannula sleeve 202 during the surgical procedure. Sleeve 202 may be formed of stainless steel or other rigid materials such as a polymeric material or the like. Sleeve 202 may be clear or opaque. The diameter of sleeve 202 may vary, but, typically ranges from about 10 mm to about 15 mm for use with the seal assembly 100 of the present disclosure.

Cannula housing 204 includes port opening 214 and luer fitting 216 positioned within the port opening 214. Luer fitting 216 is adapted for connection to a supply of insufflation gaseous is conventional in the art and incorporates valve 218 to selectively open and close the passage of the luer fitting 216. Cannula housing 204 further includes duckbill or zero closure valve 220 which tapers distally and inwardly to a sealed configuration. Closure valve 220 defines slit 222 which opens to permit passage of the surgical instrumentation and closes in the absence of the instrumentation. Closure valve 220 is preferably adapted to close upon exposure to the forces exerted by the insufflation gases in the internal cavity. Other zero closure valves are also contemplated including single or multiple slit valve arrangements, trumpet valves, flapper valves, etc. Closure valve 220 rests upon internal shelf 224 of cannula housing 204 when assembled.

Cannula housing 204 includes at least one locking recess 226 preferably two recesses arranged in diametrical opposed relation. Locking recesses 226 serve to releasably secure seal assembly 100 to cannula assembly 200.

With continued reference to FIGS. 1-4, seal assembly 100 will be discussed in detail. Seal assembly 100 may be a separate component from cannula assembly 200 and, accordingly, adapted for releasable connection to the cannula assembly 200. Alternatively, seal assembly 100 may be incorporated as part of cannula assembly 200. Seal assembly 100 includes a seal housing, generally identified as reference numeral 102, and seal member 104 which is disposed within the seal housing 102. Seal housing 102 houses the sealing components of the assembly and defines the outer valve or seal body of the seal assembly 100. Seal housing 102 defines central seal housing axis “b” which is preferably parallel to the axis “a” of cannula sleeve 202 and, more specifically, coincident with the axis “a” of the cannula sleeve 202. Seal housing 102 incorporates three housing components, namely, first, second and third housing components 106, 108, 110, respectively, which, when assembled together, form the seal housing 102. Assembly of housing components 106, 108, 110 may be affected by any of the aforementioned connection means discussed with respect to cannula housing 204.

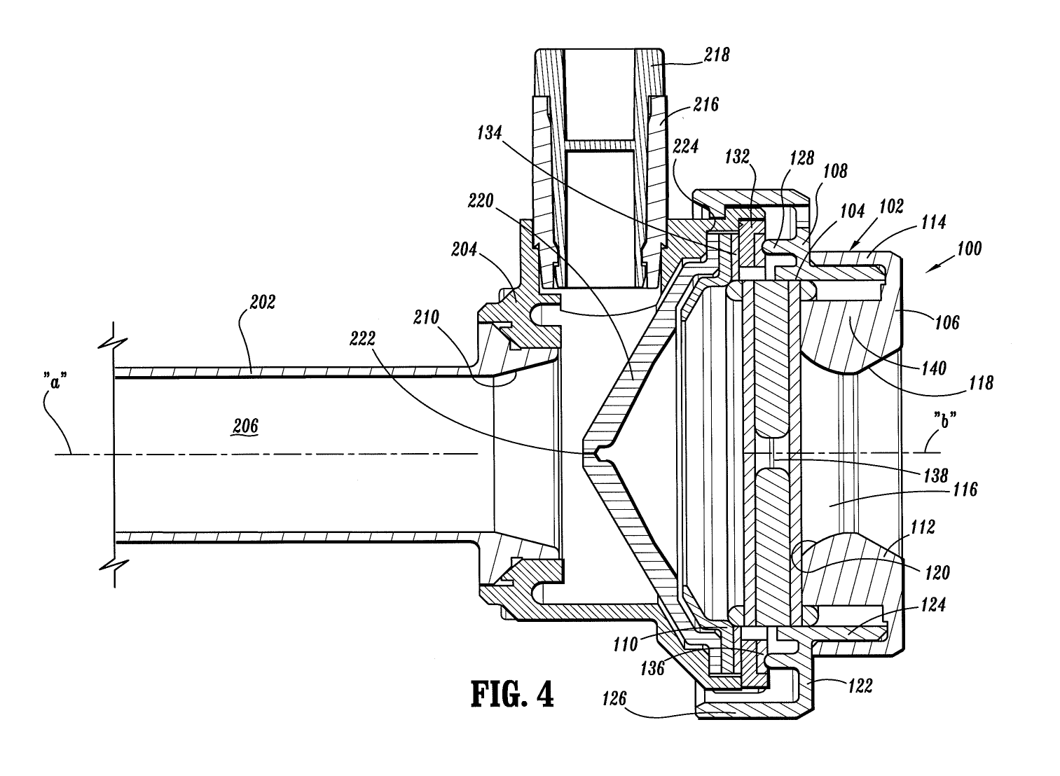

First housing component 106 defines inner guide wall 112 and outer wall 114 disposed radially outwardly of the inner guide wall 112. Inner guide wall 112 defines central passage 116 which is dimensioned to receive a surgical instrument and laterally confine the instrument within seal housing 102. As best shown in FIG. 4, inner guide wall 112 defines sloped or tapered portion 118 adjacent its proximal end. Sloped portion 118 is obliquely arranged relative to seal housing axis “b” and extends radially inwardly relative to the seal housing axis “b” in the distal direction. Sloped portion 118 assists in guiding the inserted instrument into central passage 116, particularly, when the instrument is non-aligned or off-axis relative to the seal housing axis “b”, or introduced at an angle relative to the seal housing axis “b”. Sloped portion 118 provides more flexibility to the surgeon by removing the necessity that the instrument be substantially aligned with the seal housing axis “b” upon insertion. Inner guide wall 112 is generally cylindrical in configuration and terminates in a distal arcuate or rounded surface 120.

Second housing component 108 includes transverse wall 122, inner cylindrical wall 124 depending in a proximal direction outwardly from the transverse wall 120 and outer wall 126 depending in a distal direction outwardly from the transverse wall 120. Inner cylindrical wall 124 is dimensioned to mate with outer wall 114 of first housing component 106, e.g., in a manner to be positioned within the interior of the outer wall 114 in frictional relation therewith. In the alternative, outer wall 114 of first housing component 106 may be adhered to inner cylindrical wall 124 of second housing component 108. Outer wall 126 defines scalloped outer surface 126a which is dimensioned for gripping engagement by the user.

In one embodiment, seal member 104 is mounted to proximal housing component 106 through, e.g., conventional means, such as by adhering the seal member 104 to the housing component 106 or molding the seal member 104 in the housing component 106. Seal member 104 may be fabricated from an elastomer such as a soft urethane gel, silicone gel, thermoplastic elastomer (TPE) or the like and preferably has compressible characteristics to permit the seal to receive objects having a variety of sizes, to conform and form a seal about the outer surface of the inserted object, and to close upon removal of the object. Seal member 104 may be capable of accommodating instruments of varying diameters, e.g. from about 5 mm to about 12 mm, while providing a fluid tight seal with the outer diameter of each instrument. Seal member 104 may include a central orifice 138 advantageously dimensioned to permit reception and passage of a surgical instrument. In particular, orifice 138 expands upon insertion of the surgical instrument to permit passage of the surgical instrument whereby the surface portions defining orifice 138 engage the instrument in sealed relation therewith. The orifice 138 is further adapted to assume a substantially reduced dimension upon removal of the instrument. In this position, the seal member 104 restricts the egress of gaseous matter through seal housing 102. Orifice 138 may have shapes other than that of a circular cross section opening, such as “t”-shaped, “x” shaped, helical, etc.

The use of the seal assembly 100 and cannula assembly 200 in connection with introduction of a surgical instrument will be discussed. Seal assembly 100 is mounted to cannula assembly 200 and cannula assembly 200 is introduced into an insufflated abdominal cavity. An object, e.g., an instrument, is inserted into seal assembly 100 through orifice 138 whereby the portions defining the orifice 138 stretch to accommodate the instrument diameter, as necessary. The instrument is distally passed through the valve 220 in sealed relation therewith and into the body cavity to perform the desired procedure. The instrument is removed and the orifice 138 of the seal member 104 returns to a reduced diameter configuration to assist in maintaining the integrity of the established pneumoperitoneum. Other instruments may be introduced through the seal assembly 100 and access device to perform further operative techniques as desired.

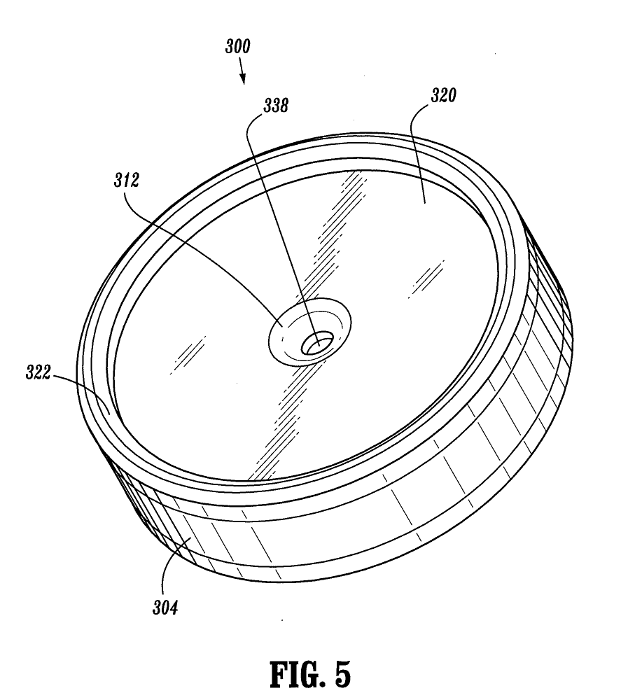

FIG. 5 illustrates a composite seal member 300 as an exemplary embodiment of a seal member in accordance with the present disclosure. Seal member 300 is configured and dimensioned to be supported within the housing of the surgical access device, e.g., between suitable surfaces of seal housing 102, and to define an access channel 312 therein. Seal member 300 includes a gel layer 304 which forms a seal with the housing interior wall and defining an orifice 338 therethrough. A fabric layer 320 sits above and below gel layer 304 such that a surface 320 of the gel layer 304 which defines orifice 338 is covered by fabric layer 320. Access channel 312 is configured and dimensioned such that insertion of a surgical instrument into access channel 312 causes seal member 300 to form a substantial sealing relation with the surgical instrument inserted therethrough.

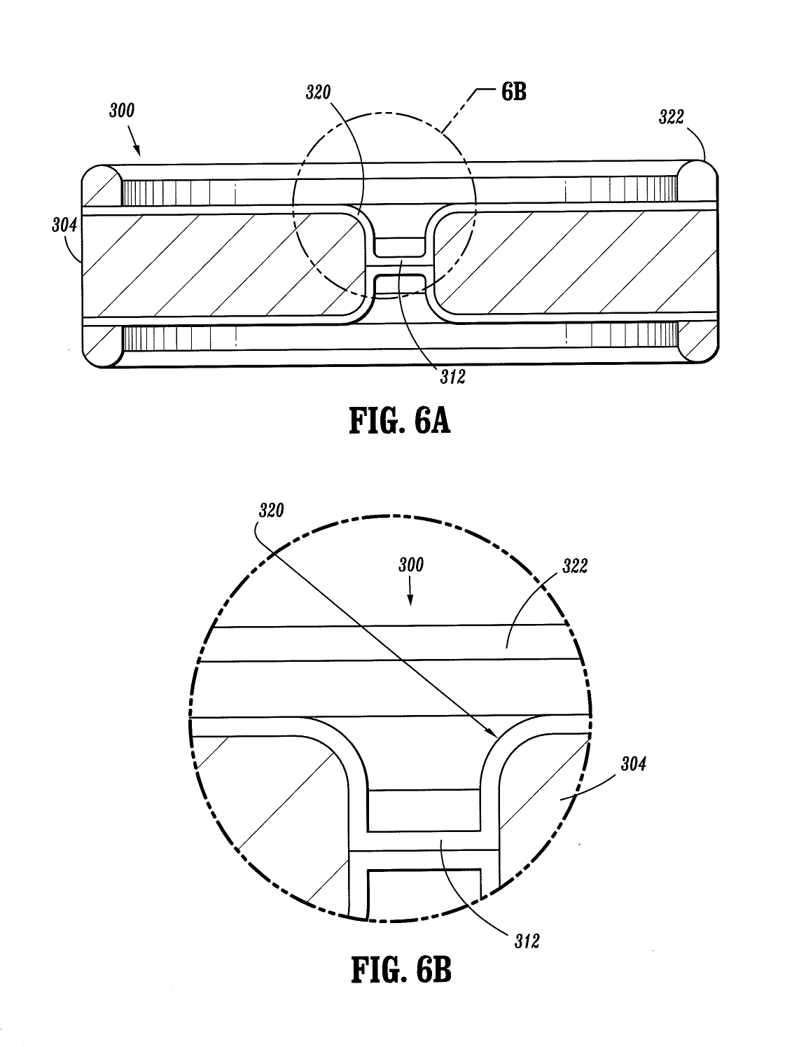

Referring now to FIGS. 6A and 6B, composite seal member 300 includes a seal assembly fabricated from a first generally soft gel layer 304 and an elastic fabric layer 320 which substantially encapsulates gel layer 304. FIG. 6B is an exploded view of the central portion of seal member 300 illustrating fabric layer 320 substantially encapsulating gel layer 304 and wherein fabric layer 320 also covers the surface of gel layer 304 which defines orifice 338.

In one embodiment, gel layer 304 may be any suitable material identified hereinabove in connection with the embodiment of FIGS. 1-4, including, for example, a thermoplastic elastomer (TPE) or other flexible lubricous material, urethane gel, a silicon gel, alginates, gum Arabic, polymer hydrogels or a polymeric thereof, or any combination of these materials. Fabric layer 320 may include a SPANDEX® material containing 20% LYCRA® available from Milliken. Alternatively, fabric layer 320 may be disposed on just one of either the proximally facing surface or the distally facing surface of seal member 300, as desired (not shown).

Fabric layer 320 provides a degree of rigidity to gel layer 304 and may desirably assist in maintaining gel layer 304 in its disc-shaped configuration. Moreover, the combination of fabric layer 320 and gel layer 304 defines a seal having enhanced adaptability to a variety of different diameter objects, e.g., instruments, and which maintains a seal upon offset manipulation of the object. Fabric layer 320 also serves as a secondary seal supplemental to the sealing functions of gel layer 304.

In still further embodiments, the seal member may include a coating to reduce frictional forces on the surgical instrument. The coating may be, e.g., an amorphous diamond coating, ion implantation coating, silicon or hydrogel coating, TEFLON® etc. and allows for more efficient manipulation of instrumentation through the access channel of a cannula or trocar.

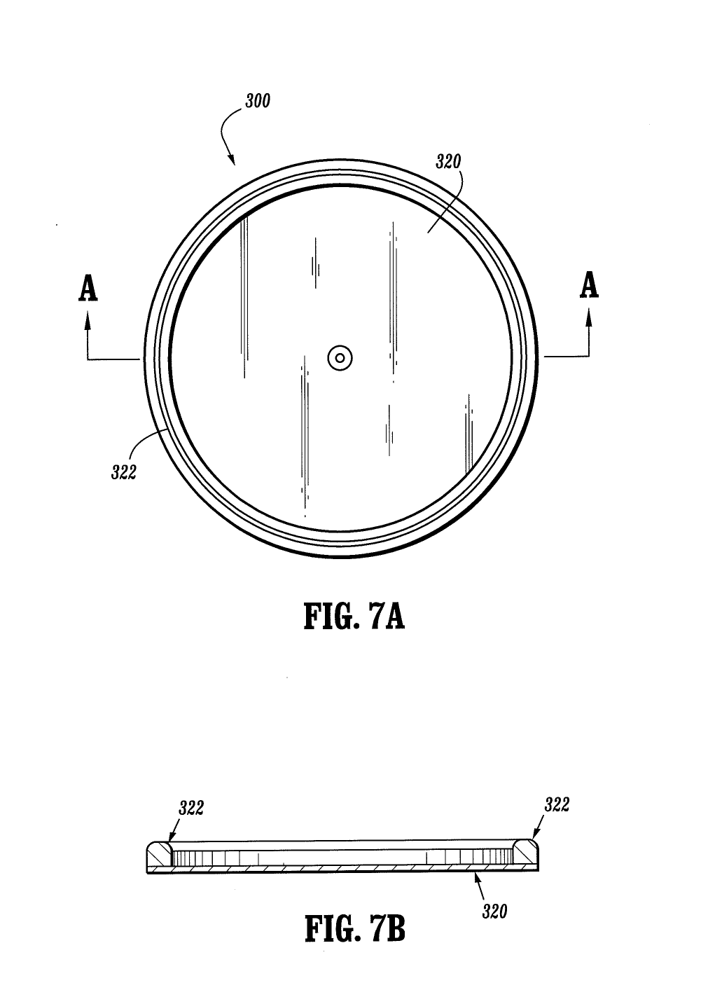

Referring now to FIGS. 7A and 7B, in one embodiment, seal member 300 illustrates a rigid ring layer 322 attached to an outer circumference of at least one of a proximal end or distal end of seal member 300. Ring layer 322 is adapted for mounting to the housing of seal member 300. Rigid ring layer 322 is made from a material such as nylon or any other rigid thermoplastic, e.g., polypropylene, polyethylene, polycarbonate, etc., and provides for a method for fixing the seal within a housing of a surgical access device.

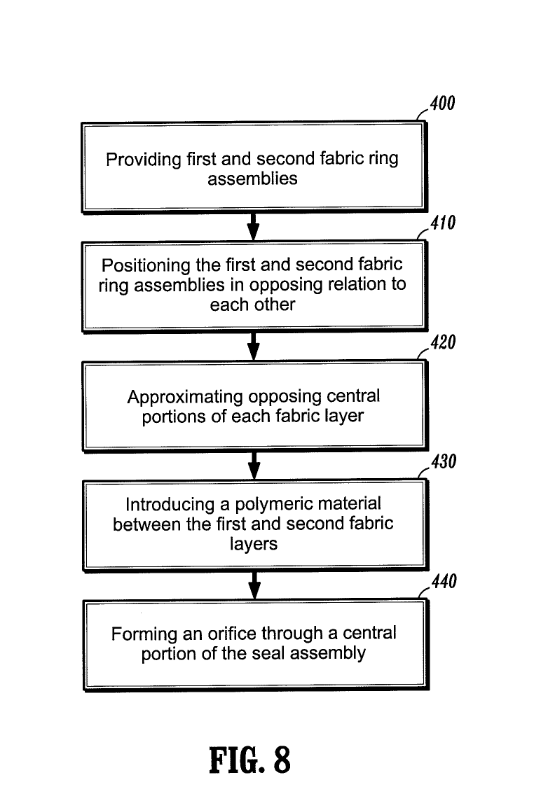

In one embodiment of a method of forming a composite seal assembly for use in a surgical access device as illustrated in the flow chart of FIG. 8, includes the step of initially providing first and second fabric ring assemblies each including a rigid ring having a fabric layer secured thereto (Block 400). The first and second fabric ring assemblies are then positioned in opposing relation to each other such that a gap is created therebetween (Block 410). Thereafter, opposing central portions of each fabric layer are approximated (Block 420) prior to introducing a gel material between the first and second fabric layers to fill the gap formed between the first and second fabric ring assemblies which forms the seal assembly (Block 430). An orifice is then formed through a central portion of the seal assembly such that the fabric layer of one or both of the fabric ring assemblies covers the surface of the orifice (Block 440).

In more detail, in some embodiments, a composite seal assembly for use in a surgical access device is formed by securing a first fabric ring assembly to a first fabric layer and a second fabric ring assembly to a second fabric layer via overmolding the fabric ring assemblies onto the fabric layers. Additionally, excess fabric on the orifice of the seal member is removed. The first and second fabric ring assemblies are then positioned in opposing relation to each other such that a gap is created therebetween via pressing the fabric ring assemblies into recesses of a mold.

The opposing central portions of each fabric layer are then pinched together with mating core pins creating an access channel for instruments to pass therebetween having good sealing properties. A thin layer of fabric at the central portion of the seal assembly is thereafter removed to form the orifice 324 which defines the access channel 312 such that the orifice accepts surgical instruments therethrough. In further embodiments, a coating is applied to the seal member to reduce frictional force with the surgical instruments. The coating may be, e.g., an amorphous diamond coating, ion implantation coating, silicon or hydrogel coating , TEFLON® coating etc., and allows for more efficient manipulation of instrumentation through the access channel of a cannula or trocar.

While the above description contains many specifics, these specifics should not be construed as limitations on the scope of the disclosure, but merely as exemplifications of embodiments thereof. Those skilled in the art will envision many other possibilities within the scope and spirit of the disclosure as defined by the claims appended hereto.

What is claimed is:

1. A surgical access device, comprising:
a housing having an interior wall defining a longitudinal axis and having at least one aperture configured and dimensioned to permit passage of a surgical instrument therethrough, a seal member supported in the housing and defining an access channel therein, the seal member including:
a resilient layer forming a seal with the housing interior wall and defining an orifice therethrough; and
a fabric layer substantially encapsulating the resilient layer such that a surface of the resilient layer which defines the orifice is covered by the fabric layer;
wherein the access channel is configured and dimensioned such that insertion of a surgical instrument into the access channel causes the seal member to form a substantial sealing relation with the surgical instrument inserted therethrough.

2. The surgical device as in claim 1, wherein the seal member further comprises a rigid ring layer attached to an outer circumference of at least one of a proximal end or distal end of the seal member.

3. The surgical access device as in claim 2, wherein the rigid ring layer is adapted for mounting to the housing.

4. The surgical access device as in claim 1, wherein the resilient layer comprises a thermoplastic elastomer.

5. The surgical access device as in claim 4, wherein the thermoplastic elastomer comprises a gel.

6. The surgical access device as in claim 1, wherein the fabric material comprises spandex.

7. The surgical access device as in claim 1, wherein the surgical access device is a cannula.

8. The surgical access device as in claim 2, wherein the rigid ring layer includes a material selected from the group consisting of nylon, polypropylene, polyethylene or polycarbonate.

9. The surgical device as in claim 1, wherein the seal member further includes a coating to reduce frictional forces on the surgical instrument.

10. The surgical device as in claim 9, wherein the coating is selected from the group consisting of an amorphous diamond coating, a coating having ion implantation, silicon coating, hydrogel coating or TEFLON® coating.

11. A method of forming a composite seal assembly for use in a surgical access device, comprising the steps of:
providing first and second fabric ring assemblies each including a rigid ring having a fabric layer secured thereto;
positioning the first and second fabric ring assemblies in opposing relation to each other such that a gap is created therebetween;
approximating opposing central portions of each fabric layer;
introducing a gel material between the first and second fabric layers to fill the gap formed between the first and second fabric ring assemblies to form the seal assembly; and
forming an orifice through a central portion of the seal assembly.

12. The method of forming a composite seal assembly for use in a surgical access device as in claim 11, wherein the step of forming an orifice through a central portion of the seal assembly further includes covering the surface of the orifice with the fabric layer of one or both of the fabric ring assemblies.

13. The method of forming a composite seal assembly for use in a surgical access device as in claim 11, wherein the providing step includes securing a first fabric ring assembly to a first fabric layer and a second fabric ring assembly with a second fabric layer.

14. The method of forming a composite seal assembly for use in a surgical access device as in claim 11, wherein the providing step further includes overmolding at least one of the first and second fabric ring assembly onto the fabric layer.

15. The method of forming a composite seal assembly for use in a surgical access device as in claim 11, wherein the securing step includes removing excess fabric from the orifice.

16. The method of forming a composite seal assembly for use in a surgical access device as in claim 11, wherein the positioning step includes pressing the fabric ring assemblies into recesses of a mold.

17. The method of forming a composite seal assembly for use in a surgical access device as in claim 11, wherein the approximating step includes pinching opposing central portions of each fabric layer with mating core pins.

18. The method of forming a composite seal assembly for use in a surgical access device as in claim 11, wherein the forming step includes removing a thin layer of fabric at the central portion of the seal assembly to uncover the surface of the orifice such that the orifice accepts surgical instruments therethrough.

19. The method of forming a composite seal assembly for use in a surgical access device as in claim 11, further comprising the step of applying a coating to the seal member to reduce frictional force with the surgical instruments.

20. The method of forming a composite seal assembly for use in a surgical access device as in claim 19, wherein the coating is selected from the group consisting of an amorphous diamond coating, a coating having ion implantation, silicon coating, hydrogel coating or TEFLON® coating.
